(19) United States
(12) Patent Application Publication
(10) Pub. No.: US 2023/0405591 A1
(43) Pub. Date: Dec. 21, 2023

(54) MULTI-CHANNEL INTEGRATED MICROFLUIDIC CHIP AND METHOD FOR HIGH-THROUGHPUT PREPARATION OF MONODISPERSE MICROGELS USING THE SAME

(71) Applicant: DALIAN UNIVERSITY OF TECHNOLOGY, Dalian, Liaoning (CN)

(72) Inventors: Huanan WANG, Dalian, Liaoning (CN); Haoyue ZHANG, Dalian, Liaoning (CN); Chuanfeng AN, Dalian, Liaoning (CN)

(21) Appl. No.: 18/249,801

(22) PCT Filed: May 19, 2021
(86) PCT No.: PCT/CN2021/094524

§ 371 Date: Apr. 20, 2023

(30) Foreign Application Priority Data
Oct. 20, 2020 (CN) 202011126744.X

(51) Int. Cl. B01L 3/00

(52) U.S. Cl. CPC B01L 3/502746; B01L 2200/10; B01L 2300/0883

(57) ABSTRACT

A multi-channel integrated microfluidic chip has at least two layers of channel structures. Each layer of channel structure is provided with a liquid phase input channel. One layer of channel structure is provided with a drop-maker unit and a collection channel. The liquid phase input channel has a liquid phase input port (1) and a resistance control unit (2). The drop-maker unit contains a multiphase emulsification channel (6) and a local resistance control unit (8). The collection channel has a washing channel (9), a washing phase input port (10) and a product output port (11). A method for preparing monodisperse gel microspheres is also provided.

### TECHNICAL FIELD

The present invention relates to the technical field of bioengineering, and particularly to a multi-channel integrated microfluidic chip and a method for high-throughput preparation of monodisperse microgels by using the same.

### BACKGROUND ART

Droplet microfluidics technology is a microfabrication technology based on a microfluidic chip for precisely controlling immiscible multiphase fluids, and is capable of continuous sample injection and rapid production of monodisperse microgels or microcapsules with precise size control. Compared with the traditional water-in-oil (W/O) or oil-in-water (O/W) single emulsion droplet technology, the droplet-based microfluidic technology may prepare single emulsion droplets in uniform size by a microfluidic device with a T-junction or a flow-focusing channel structure. Moreover, this can be used as a template to prepare monodisperse microgels through different polymerization modes. However, like the traditional emulsion method, the single-emulsion droplet-based microfluidic technology is unsuitable for continuous processing of microgels carrying bioactive substances. This is because that: 1) the traditional microfluidic technology is also based on emulsion droplets, which require continuous production and preparation of emulsion droplets, and then the hydrogel prepolymers in the droplets are solidified to obtain microgels; however, in the process of preparing samples, immobilized active substances are exposed to an oil phase, a surfactant, a cross-linking agent and the like for a long time to cause material toxicity; 2) moreover, droplets containing microparticles need to be collected in the process of preparation for washing in a second step, the oil phase and the surfactant need to be cleaned in different ways, such step is not only time-consuming and labor-intensive, and the carried substances in the microparticles cannot be subjected to substance exchange with the outside before washing; and 3) the productivity of traditional single-channel microfluidic technology is about 10⁴ droplets per second (that is, 10⁷-10⁸ droplets per hour), while the flow rate of the internal phase aqueous solution is 0.3-1 ml per hour, the productivity thereof is still far from meeting the requirements of biomedical applications (Liu, H. et al. Advances in Hydrogel-based Bottom-Up Tissue Engineering. SCIENTIA SINICA Vitae45, 256-270).

Practical application of cell-carried microgels is taken as an example. As a basic component of modular assembly engineering, cell-carried microgels have promising application prospects in single-cell behavior studies and tissue 3D printing. At the current laboratory level, the droplet-based microfluidic technology based on a single flow-focusing drop-maker unit is capable of easily achieving the preparation of a small amount of monodisperse-carried single-cell microgels through liquid phase flow and channel size control, the cell activity has been also very considerable, and a certain progress has been made in the subsequent induction of cell differentiation and in-vivo implantation (Choi, C. H. et al. One-step generation of cell-laden microgels using double emulsion drops with a sacrificial ultra-thin oil shell. Lab Chip 16, 1549-1555; Zhang, L. et al. Microfluidic Templated Multicompartment Microgels for 3D Encapsulation and Pairing of Single Cells. Small 14). However, the production efficiency of the existing microfluidic cell immobilization technology remains a major bottleneck. Due to the cell needs to be prevented from being damaged by shear force generated by high flow velocity in the process of microfluidic cell immobilization, the productivity of the existing single-channel microfluidic technology is generally 10³-10⁴ droplets per second (that is, 10⁷-10⁸ droplets per hour), and the amount of cell suspension that can be processed per hour is about 0.3-1 mL. However, human tissues generally have a cell density more than 10⁸ cells/mL, which means that the construction of a 1 mL-volume of tissue-like tissue with single-cell immobilized microgels as the basic unit requires more than 10 hours of continuous microgel microfluidic production. On the other hand, in terms of clinical cell therapy application, each administration dosage is on the order of 10⁸-10⁹ cells, which means that the preparation of cell capsules of such an order requires more than 10 hours of continuous microgel microfluidic production. Such production efficiency greatly limits the practical clinical application of microfluidic technology for cell immobilization.

Since the single-channel microfluidic droplet technology is limited by the productivity, how to improve the throughput of microfluidic droplet technology has become an important issue in the art. Based on existing microfluidic droplet technology for producing micro-droplets using a single drop-maker unit, for microfluidic amplification technology, some progress has been made in the development of high throughput production technology by integrating a great number of drop-maker units in recent years. By improving the size of the overall channel, Femmer et al. significantly reduced the fluid resistance of the overall channel, realized the integration of a certain number of drop-maker units, and achieved high throughput production of large-sized droplets (T. Femmer, A. Jans, R. Eswein, N. Anwar, M. Moeller, M. Wessling, A. J. Kuehne, High-Throughput Generation of Emulsions and Microgels in Parallelized Microfluidic Drop-Makers Prepared by Rapid Prototyping. ACS Appl Mater Interfaces, 2015, 7(23), 12635-8). Jeong et al. adopted a liquid phase distribution channel with a channel cross-sectional area much greater than that of the drop-maker unit to significantly reduce the difference in flow distribution caused by liquid phase distribution, and greatly improve the integration of the drop-maker unit, and achieved a micro-droplet yield up to 7.3 liters per hour in a high-precision processed glass-monocrystalline silicon chip (Yadavali, S., Jeong, H. H., Lee, D. & Issadore, D. Silicon and glass very large-scale microfluidic droplet integration for terascale generation of polymer microparticles. Nat Commun 9, 1222). According to Nisisako et al., an annular arrangement integrated chip was selected, and a distribution was carried out in a disc or ring shape to achieve equalization of channel resistance before entering each channel, thereby realizing uniform flow distribution, and further realizing a large number of integration of drop-maker units and high-throughput production of emulsion droplets (Nisisako, T., Ando, T. & Hatsuzawa, T. High-volume production of single and compound emulsions in a microfluidic parallelization arrangement coupled with coaxial annular world-to-chip interfaces. Lab Chip 12, 3426-3435). According to Conchouso et al, a symmetrical branching mode was adopted in the liquid distribution and collection channels while a circular arrangement was used, which reduces errors and avoids the problem of clogging to some extent in open channels such as discs or rings, and significantly improves the stability of integrated production devices (D. Conchouso, D. Castro, S. A. Khan, I. G. Foulds, Three-dimensional parallelization of microfluidic droplet generators for a litre per hour volume production of single emulsions. Lab Chip, 2014, 14(16), 3011-3020).

However, these high-throughput methods above are basically based on the design philosophy of uniform convey in wide channel and distribution after entering the narrow channels. For the design based on such philosophy, the higher fluid resistance of the narrow channel is used to balance the resistance difference caused by the distribution of wide channel, thereby maintaining the consistent flow pattern among drop-maker unit s. However, after the cells are introduced, a relatively low flow velocity in the wide channel and the switching structure between the wide and narrow channels easily lead to the accumulation of various particles (cells, cell debris, microgels) in the liquid phase, so as to induce clogging and result in the problem of difference in flow pattern among different channels, which mechanistically unable to meet the microparticle system carried by the particles. By adopting symmetrical branching mode of equal-sized channels to reduce the flow difference between channels, Headen et al. achieved an expanded preparation of cell-carried microgels in a device integrating eight channels, but the maximum yield of 0.6 mL per hour thereof remains unable to meet the requirements of tissue engineering, cell therapy, and cell 3D printing (D. M. Headen, J. R. Garcia, A. J. Garcia, Parallel droplet microfluidics for high throughput cell encapsulation and synthetic microgel generation. Microsystems & Nanoengineering, 2018, 4(1)).

The other type of high-throughput production method that adopts step emulsification is subject to a basic principle that Laplace pressure difference is induced when a two-phase interface passes through the channel geometry in a quasi-static state, and droplets are thus formed spontaneously. This method reduces the correlation between the particle size of the formed droplets and the liquid phase flow velocity (only an upper limit of the flow velocity), thereby greatly avoiding the problem of uneven flow distribution generated during the high-throughput production of micro-droplets and ensuring that the formation process of the droplets is milder. Meanwhile, the size of droplets may be also controlled by adjusting the size and shape of the enlarged opening of the micro-channels, the hydrophilicity and the hydrophobicity of the channels and the like. Based on this principle, Amstad et al. designed a “Millipede” chip with 500 flat channels, and microdroplet particles essentially the same size was produced at a flow gradient in this series of channels arranged in parallel, ultimately achieving a droplet yield of up to 150 mL per hour (Amstad, E. et al. Robust scalable high throughput production of monodisperse drops. Lab Chip 16, 4163-4172). Stolovicki et al. introduced buoyancy to further simplify the conditions for droplet formation, which greatly simplifies the structure of the droplet production device and the processing difficulty thereof, and simultaneously further expands the particle size range of the products (E. Stolovicki, R. Ziblat, D. A. Weitz, Throughput enhancement of parallel step emulsifier devices by shear-free and efficient nozzle clearance. Lab Chip, 2017, 18(1), 32-138.). Based on this principle, Huang et al. also adopted a side-by-side glass tubes to suspend droplets, considered the gravity factor caused by density difference while droplets are formed based on interfacial force, further simplified the conditions for stable formation of droplets, and also achieved continuous production of droplets with Janus structure (X. Huang, M. Eggersdorfer, J. Wu, C.-X. Zhao, Z. Xu, D. Chen, D. A. Weitz, Collective generation of milliemulsions by step-emulsification. RSC Advances, 2017, 7(24), 14932-14938).

However, for the preparation requirements of cell-carried microgels, the required range on physical parameters (such as flow velocity and liquid phase viscosity) of the step emulsification remains too narrow. Since each liquid phase is subject to an upper limit of the number of capillaries (related to the viscosity, flow velocity and density of the liquid phase) in the production process, the excessive viscosity of the hydrogel prepolymer itself will further limit the upper limit of the flow velocity in a single channel. When the cell-carried microgels are produced at an excessively low liquid phase flow rate, the probability of cell sedimentation and aggregation is significantly improved, which seriously affects the product quality.

In summary, the existing chip design for the high-throughput microfluidic droplet technology is oriented towards the preparation of polymer microspheres or microspheres of simple material systems, rather than towards the immobilization of living cells with biological activity or bioactive protein drug molecules, therefore, it is unnecessary to consider harsh conditions for embedding bioactive substance for the chip design, including: 1) the micro-channels are easy to be clogged when living cells are used as immobilized substances; 2) when more parallel channels are introduced or a high-viscosity hydrogel prepolymer or a monomer solution is taken as a disperse phase, it is easy to make the size of prepared droplets or gel microspheres nonuniform; 3) when living cells or bioactive protein molecules are immobilized, the high-throughput microfluidic production conditions deliver an impact on the biological activity of the immobilized substances; and 4) the droplets or microgel production device are hard to run stably for a long time under complex preparation conditions. In summary, to design and prepare the microfluidic chip technology for high-throughput preparation of droplets or microgels for immobilizing bioactive substances and to make the same suitable for immobilization of living cells or bioactive protein drug molecules are key issues to break through the application of microparticle carrying bioactive substances in clinical or other fields.

### SUMMARY

In order to solve the problems in the prior art, it's an object of the present invention to provide a multi-channel integrated microfluidic chip to ensure continuous and stable high-throughput production of cell-carried microgels in the chip, and to complete the demulsification separation in the chip.

In order to solve the technical problems above, the present invention uses the following technical solution:

A multi-channel integrated microfluidic chip, including at least two layers of channel structure, at least two liquid phase input channels, at least two drop-maker units and a collection channel; where each layer of channel structure is provided with a liquid phase input channel, one of the layers of channel structure is provided with a drop-maker unit, and the collection channel is contained in one of the layers of channel structure or runs through the multiple layers of channel structure; each liquid phase input channel includes at least one liquid phase input port, the liquid phase input port is connected to at least one resistance control unit, and each resistance control unit corresponds to one output port.

The drop-maker unit includes an input port, a liquid phase input channel, an emulsification channel, an output channel and a local resistance control unit, where the output ports on the different layers of channel structure correspond to input ports on the same and are communicated with each other through a microfluidic channel, and the resistance control units on the same layer of channel structure are directly connected to the emulsification channel.

The collection channel includes a washing channel, a washing phase input port and a product output port.

In the above technical solution, further, when the number of input liquid phases is 2, the two liquid phases are input through the liquid phase input ports on the upper and lower surfaces of the chip, respectively; when the number of the input liquid phases is greater than or equals to 3, the sample injection ports of the input liquid phases at the layers other than the uppermost and the lowermost layers are respectively connected to the side surfaces of the chip through the horizontal input channels to input liquid phases.

There are at least two drop-maker units, and each of the drop-maker unit is directly connected to all the liquid phase input channels and the collection channels.

In the above technical solution, further, a structure of the resistance control unit is selected from one or a combination of some of a mesh groove, an annular groove and an S-shaped channel, and a structure of the local resistance control unit is selected from one or some of a local bayonet structure, an S-shaped channel structure or an enlarged cavity structure. Different fluid characteristics correspond to different resistance control structures respectively, so that the objective of reducing the production power consumption while balancing flow resistance is achieved.

In the above technical solution, further, a structure of the emulsification channel in the drop-maker unit is selected from one or some of a flow-focusing structure, a T-junction structure, a co-flow structure, a Y-junction structure, a three-branch structure and a four-branch structure.

In the above technical solution, a channel cross-sectional area of the drop-maker unit of the chip is 25 μm²-10⁶ μm².

In the above technical solution, further, the washing channel is annularly arranged in a unidirectional way, output channels of all the drop-maker units are equidistantly arranged on the inner circumference of the washing channel, the washing channel covers all the output channels, the beginning and the end of the washing channel are a washing phase input port and a product output port, respectively, and each corner is rounded to prevent local flow dead ends.

In order to achieve a uniform distribution of the liquid phase in each drop-maker unit, the fluid resistance of each channel structure in the chip needs to be determined after relevant balance calculation. The formula for calculating the fluid resistance of a square microchannel is as follows:

R=12(μL/wh³)(1−0.63h/w)⁻¹

where R is the channel fluid resistance, μ is the channel resistance coefficient, L is the channel length, w is the channel width, and h is the channel height.

The literature (Romanowsky, M. B., Abate, A. R., Rotem, A., Holtze, C. & Weitz, D. A. High throughput production of single core double emulsions in a parallelized microfluidic device. Lab Chip 12, 802-807) indicates that, in order to ensure equal flow velocity of all liquid phases in all drop-maker units, the flow attenuation generated by the liquid phase distribution channel and the collection channel needs to be reduced to a negligible level, that is, the fluid resistance of the liquid phase distribution channel and the collection channel needs to be much smaller than that in the unidirectional channel to which the corresponding drop-maker unit belongs (namely, R_{c}<Rᵤ), overall, satisfying:

Sum(R_{c})/Rᵤ<0.01

where Sum(R_{c}) is the sum of the fluid resistances of the liquid-phase distribution channel and the washing channel, and Rᵤ is the total fluid resistance in the unidirectional channel to which the corresponding drop-maker unit belongs, and its specific distribution is shown in FIG. 21C.

By combining with the above resistance calculation formula and the design requirements on the integrated channel, the channel cross-sectional area of the washing channel needs to be more than 10 times of that of the drop-maker unit, so as to greatly reduce the fluid resistances of the liquid phase distribution channel and the collection channel, and prevent the microgels from clogging the channel.

In the above technical solution, further, the liquid phase input module and the drop-maker units in the chip are arranged in a centrosymmetric manner with the sample injection port as the center, and the size of the drop-maker unit is much smaller than that of the washing channel, so as to achieve the objective of eliminating the resistance difference between different channels during the liquid phase input, and sample injection ports of all liquid phase input modules are located on the same longitudinal axis. The distances between the sample injection port and the sample outlet port on the same substrate layer are equal.

In the above technical solution, further, the vertical distances from the emulsification channel to the washing channel of each drop-maker unit are equal.

In the above technical scheme, further, for different liquid phase systems, the channels are required to be subjected to affinity treatment on the whole, that is, inner surfaces of all the channels are coated with specific affinity coatings.

On the other hand, the present invention provides a method for preparing monodisperse gel microspheres. The method uses the aforesaid microfluidic chip, a single or multiple dispersion phases are used as a first fluid, a continuous phase is used as a second fluid, and a washing phase is used as a third fluid; the first fluid and the second fluid enter the emulsification channel in the drop-maker unit through the liquid phase input channel, the first fluid is sheared by the second fluid in the emulsification channel to form droplets and then form microgels to enter a washing output module; when the number of the liquid phase of the first fluid is greater than or equal to 2, all the liquid phases are combined into one phase in the channel and then enter the emulsification channel; the third fluid cleans the two-phase emulsion in a washing module, the flow velocity in the washing module is maintained to prevent micro gel particles from aggregating and clogging, and droplets of the first fluid form the monodisperse gel microspheres through an internal crosslinking of macromolecules.

In the above technical solution, further, the first fluid is a bioactive substance suspended in the dispersed phase; when multiple carrying is performed, the carrying method of different substances is selected from one of suspended in the same dispersed phase, suspended in a plurality of groups of pre-differentiated dispersed phases, suspended in a plurality of groups of dispersed phases difficult to be mutually soluble in a same solvent, and suspended in a mutually soluble multi-dispersed phases, wherein the bioactive substances are selected from one or more of living cells, drugs, nucleic acids, proteins, flavors, nanoparticles and quantum dots.

A carrier macromolecule in the first fluid comprises one or more of a hydrogel prepolymer and a crosslinkable macromolecule prepolymer; a curing manner of the prepolymer in the first fluid comprises one or more of chemical crosslinking, photo-crosslinking, temperature-sensitive curing and phase separation.

The second fluid comprises at least one surfactant.

At least one phase of the first fluid, the second fluid and the third fluid contain at least one prepolymer crosslinking initiator. A crosslinking initiator is not needed when the temperature-sensitive curing is adopted.

When the preparation of the cell-carried microgels is performed, the third fluid is an aqueous phase, the main body of the third fluid is a cell-compatible solvent, and also comprises a pH buffering agent.

The monodisperse gel microspheres comprise microgel particles, microcapsules/micro-vesicles and multi-cavity microcapsules, with an average particle size being greater than or equal to 5 μm.

The present invention has the following beneficial effects:

The present invention provides a multi-channel integrated microfluidic chip for preparing cell-carried microgel particles in a high-flux manner, which has the beneficial effects that:
1) In view of the problems of inevitable resistance distribution and inevitable flow attenuation among different drop-maker units in the traditional parallel design philosophy, the present invention ensures that the two-phase hydraulic pressure in the liquid-phase mixing area of each drop-maker unit tends to be consistent (the difference in hydraulic pressure <1%) through the design of a huge washing channel and the design of a high resistance channel in the drop-maker unit. Therefore, the present invention can ignore partial resistance error caused by the manufacturing process and structural design requirements under the condition of keeping the liquid phase flow velocity (1-3 m/s), and ensure uniform distribution of liquid phase flow among all drop-maker units under the condition of achieving high-density integration, and stable operation of multi-channels and continuous production of microgel particles with uniform particle size distribution (the coefficient of variation (CV)<4%) are realized;
2) In view of the problem of easy accumulation of particles caused by high channel size but low flow velocity in the traditional parallel design philosophy, the present invention forms a laminar boundary layer with significant difference in flow velocity in the microchannel by maintaining a relatively high liquid phase flow velocity in the channel, thereby effectively avoiding the accumulation and clogging of carried particles, so that the liquid phase channel operates in a stable and continuous manner. Meanwhile, the unidirectional introduction of the washing phase in the washing channel can further increase the flow velocity in the washing channel while the demulsification/further solidification of the emulsion is realized, thereby avoiding the accumulation of microgels in the channel and improving the stability of the production process;
3) Compared with the existing multi-step production method for microgels step by step, the present invention enables droplets to directly enter the washing channel after being output by the drop-maker unit. In the channel, when a specific emulsion formula is used, the steps of solidification, washing and separation of microgels can be integrated into the same chip by introducing a washing agent, which greatly simplifies the production process of microgels; other modification factors can be introduced into the microgels to further process the formed microdroplets/microgels, and the diversity of products is greatly improved;
4) Compared with the existing microgel production method by a branched parallel integrated drop-maker unit (the cell suspension treatment rate <0.6 ml/h) (D. M. Headen, J. R. Garcia, A. J. Garcia, Parallel droplet microfluidics for high throughput cell encapsulation and synthetic microgel generation. Microsystems & Nanoengineering, 2018, 4(1)), the present invention uses a annular parallel integrated structure with a higher channel density, thereby realizing continuous and stable high-throughput production of microgels carrying micron-sized particles (cells), and the treatment throughput of cell suspension is improved by more than two orders of magnitude (greater than 10 ml/h); in addition, in view of a hydrogel prepolymer system with a relatively low viscosity and difficult sedimentation of carried particles, the production throughput can be further improved (greater than 20 ml/h); and
5) By keeping the drop-maker units relatively independents with each other, the present invention can introduce the drop-maker units with different structures into a chip and realize the operation, so that the chip can be applicable to the production of different materials (including but not limited to various hydrogel materials, soluble plastics and resin materials), microparticles with different structures (including but not limited to multi-petal structures, multi-cavity structure and core-shell structure), and microparticles with different sizes (greater than 5 μm).

### BRIEF DESCRIPTION OF THE DRAWINGS

FIG. 1 is an overall structural schematic diagram of an integrated microfluidic chip provided in the present invention (taking the integration of 16 drop-maker units as an example),
wherein A and C—liquid phase distribution substrate, B—liquid phase production substrate, 1—liquid phase input port, 2—resistance control unit, 3—output port, 4—input port, 5—liquid phase input channel, 6—emulsification channel, 7—output channel, 8—local resistance control unit, 9—washing channel, 10—washing phase input port, 11—product output port;

FIG. 2 is a three-dimensional structural schematic diagram of an integrated microfluidic chip provided in the present invention (taking the integration of 16 drop-maker units as an example),
wherein A and C—liquid phase distribution substrate, B—liquid phase production substrate, 1—liquid phase input port, 2—resistance control unit, 3—output port, 4—input port, 5—liquid phase input channel, 6—emulsification channel, 7—output channel, 8—local resistance control unit, 9—washing channel, 10—washing phase input port, 11—product output port;

FIG. 3 is a three-dimensional pipeline schematic diagram of an integrated microfluidic chip provided in the present invention; in order to highlight the pipeline structure and the microdroplets production principle, various resistance control units are omitted, and the size ratios thereof do not represent the actual situation (taking the integration of 16 drop-maker units as an example),
wherein A and C—liquid phase distribution substrate, B—liquid phase production substrate, 1—liquid phase input port, 6—emulsification channel, 7—output channel, 9—washing channel, 11—product output port, 12—input channel;

FIG. 4 is a structural schematic diagram of different resistance control units,
wherein A—mesh groove, B—S—shaped channel, and C—annular groove;

FIG. 5 is a structural schematic diagram of different drop-maker units, not all of which include all the following structures,
wherein A—single-phase microgel drop-maker unit, B—Janus microgel drop-maker unit, C—core-shell structural microgel drop-maker unit, and D—4-petal microgel drop-maker unit (four-branch channel);

FIG. 6 is a schematic diagram of different local resistance control unit structures, not all of which include all the following structures,
wherein A—local bayonet structure, B—S-shaped channel structure, and C—enlarged cavity structure;

FIG. 7 is a real picture of integrated chips by 16 and 80 drop-maker units of the present invention, and the contrast object is a coil in CNY 1;

FIG. 8 is an electron microscope image of a local structure of the integrated chip of the present invention,
wherein A is cross-sectional views of the washing channel, enlarged cavity and drop-maker unit, B is a cross-sectional view of the enlarged cavity, C is a cross-sectional view of the drop-maker unit, D is a cross-sectional view of the S-shaped resistance control unit; 2—resistance control unit, 5—liquid phase input channel, 6—emulsification channel, 7—output channel, 8—local resistance control unit, 9—washing channel;

FIG. 9 is a diagram of actual liquid phase flow state at different locations in the chip, with drop-maker units labeled in order from the washing phase inlet as drop-maker unit #1, drop-maker unit #2 . . . drop-maker unit #16,
wherein a-i and a-ii are the droplet formation diagrams in a drop-maker unit in the actual production process under two flow patterns, b is a liquid phase flow state at the end of drop-maker unit #1, c is a liquid phase flow state at the end of drop-maker unit #8, and d is a liquid phase flow state at the end of drop-maker unit #16;

FIG. 10 shows the state of direct layering of the microparticle product prepared by the chip of the present invention;

FIG. 11 is a fluorescence image of empty chemically crosslinked microgels prepared in Example 1;

FIG. 12 is a micrograph of cell-carried microgels prepared in Example 1, with a measuring scale being 100 μm;

FIG. 13 is a structural diagram of a chip used in Comparative Example 1;

FIG. 14 is a diagram of actual liquid phase flow state of different washing channel structures at different time points;
wherein A and B are actual micrographs at the position of the circle when microgels are prepared by using a washing channel chip with a symmetrical structure at 0 minute and 15 minutes respectively, C and D are actual micrographs at the position of the circle when microgels are prepared by using a washing channel chip with a ring-shaped structure at 0 minute and 30 minutes respectively;

FIG. 15 is a diagram illustrating the formation of droplets in a drop-maker unit during the production of core-shell microgels in Example 2;

FIG. 16 is a product fluorescence image of producing core-shell structure microgels in Example 2;

FIG. 17 is a product fluorescence image of the Janus microgels in Example 3;

FIG. 18 is a micrograph of a photo-crosslinked hydrogel product in Example 4;

FIG. 19 is a particle size distribution diagram of hydrogel products produced under different flow ratio conditions in Example 6;

FIG. 20 is a particle size distribution diagram of droplet products produced under different flow ratio conditions in Example 7;

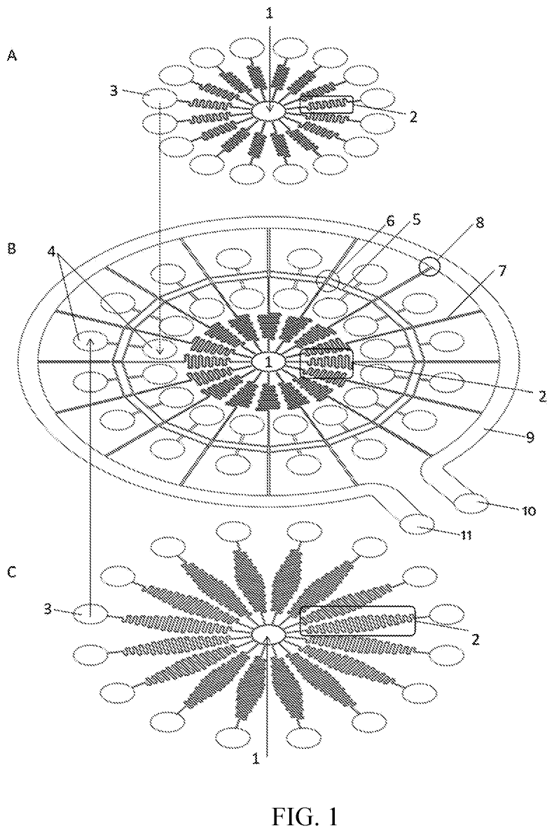

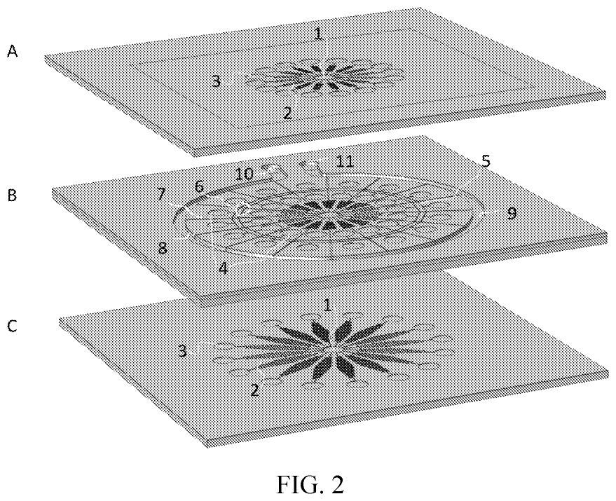

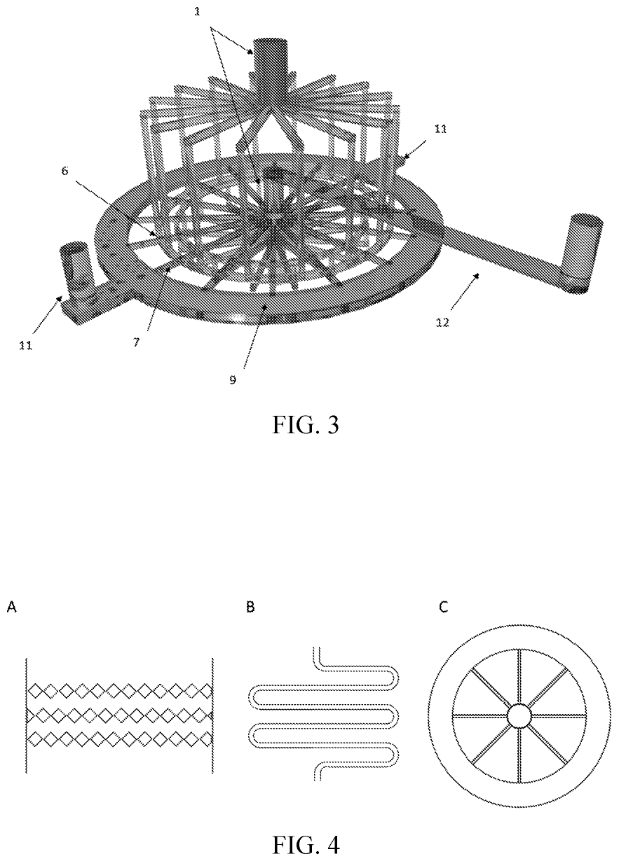

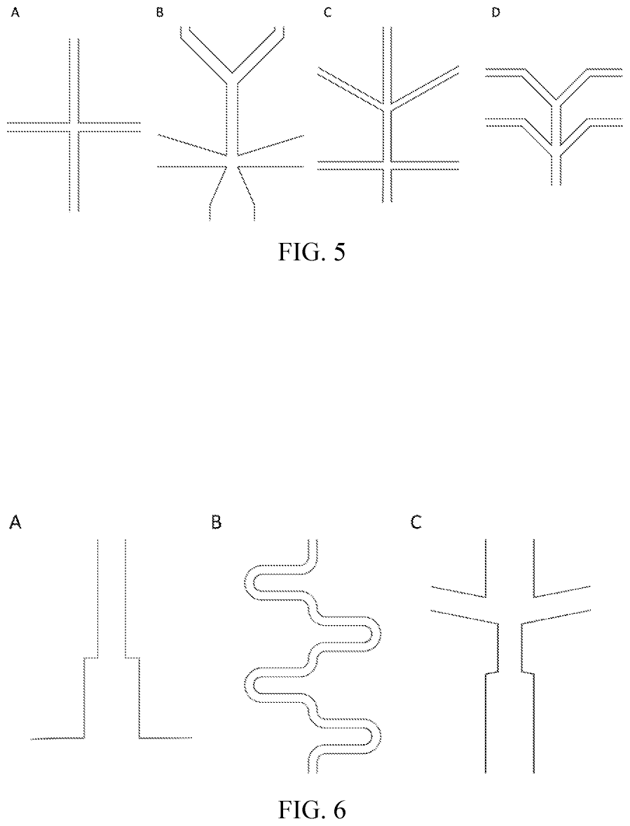

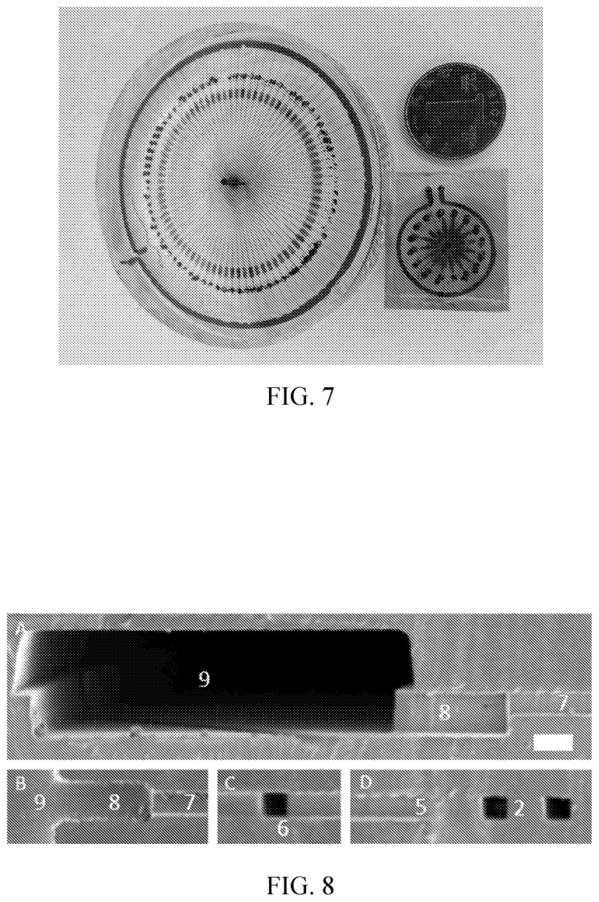

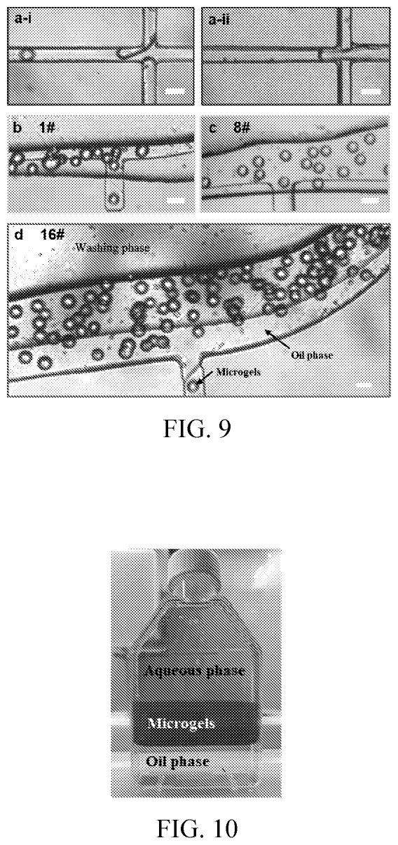

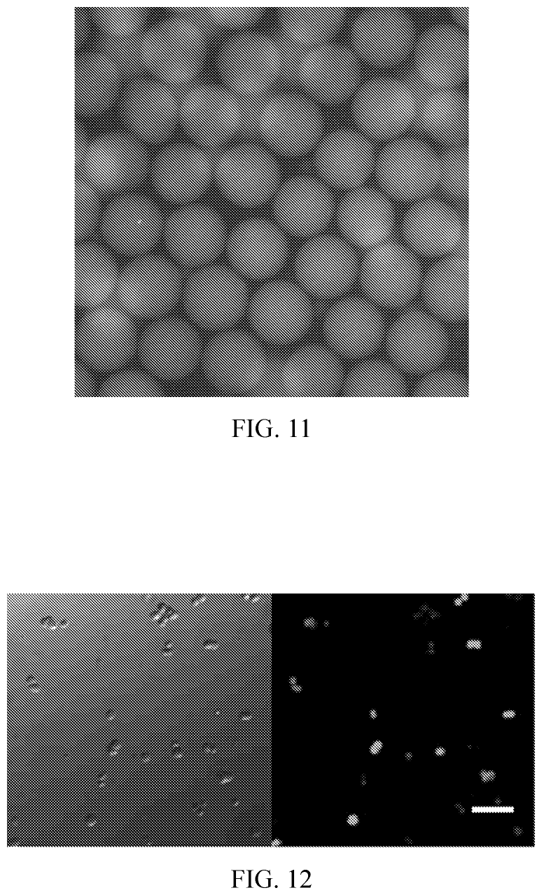

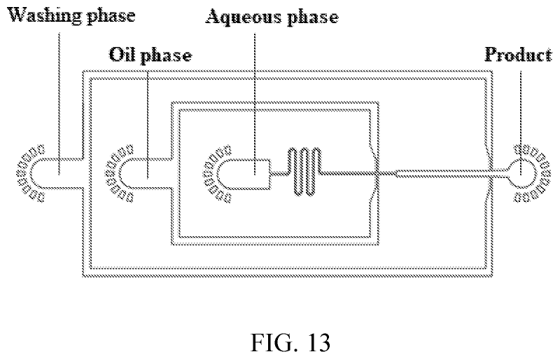

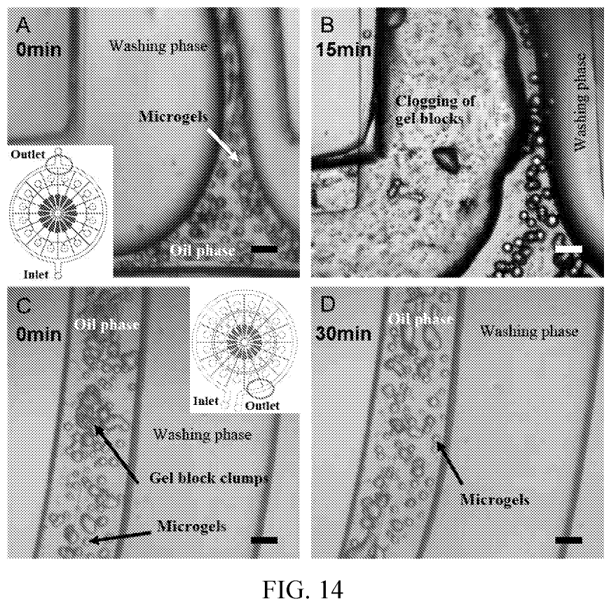

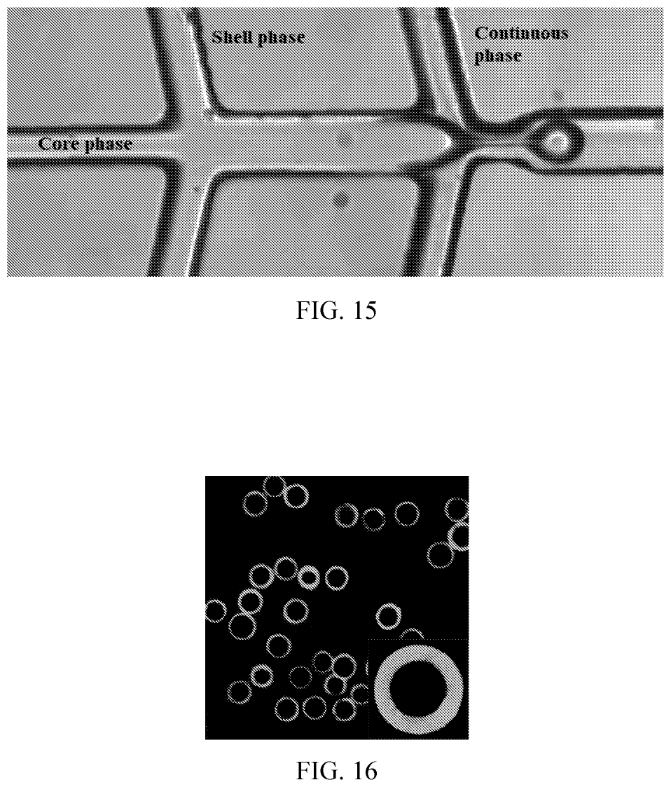

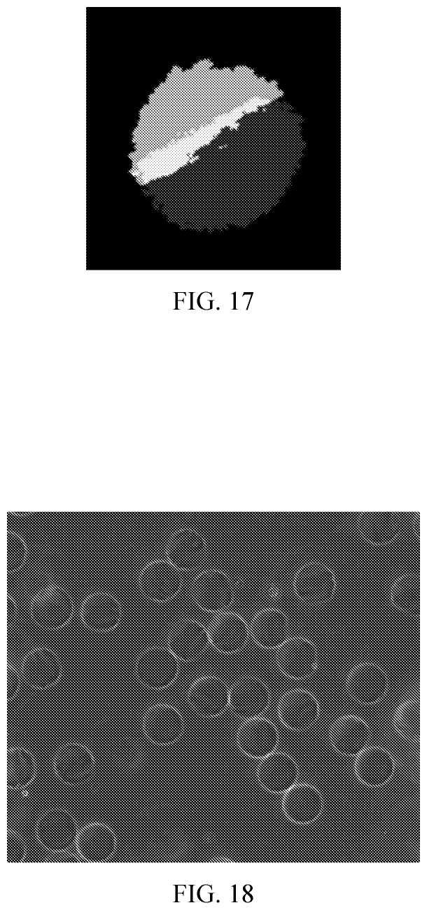

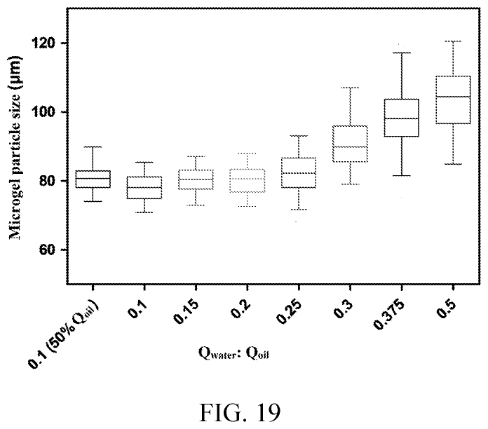

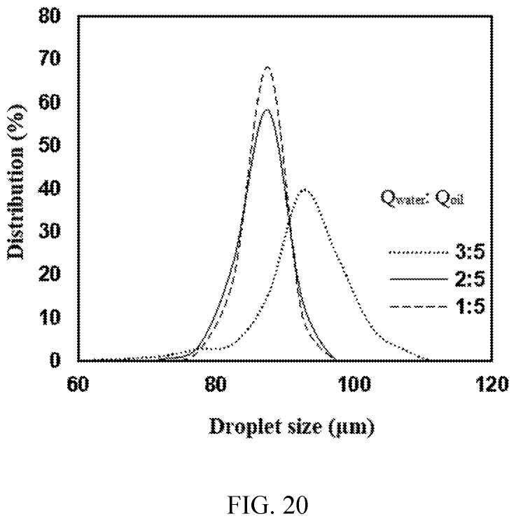

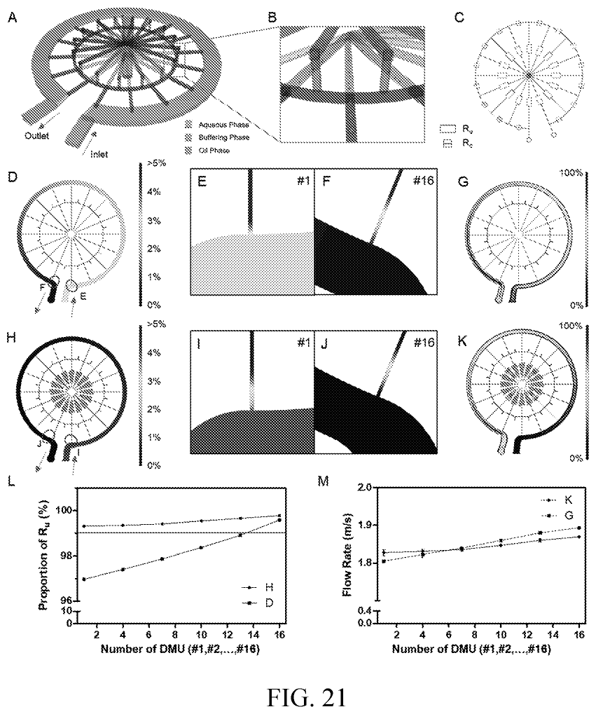

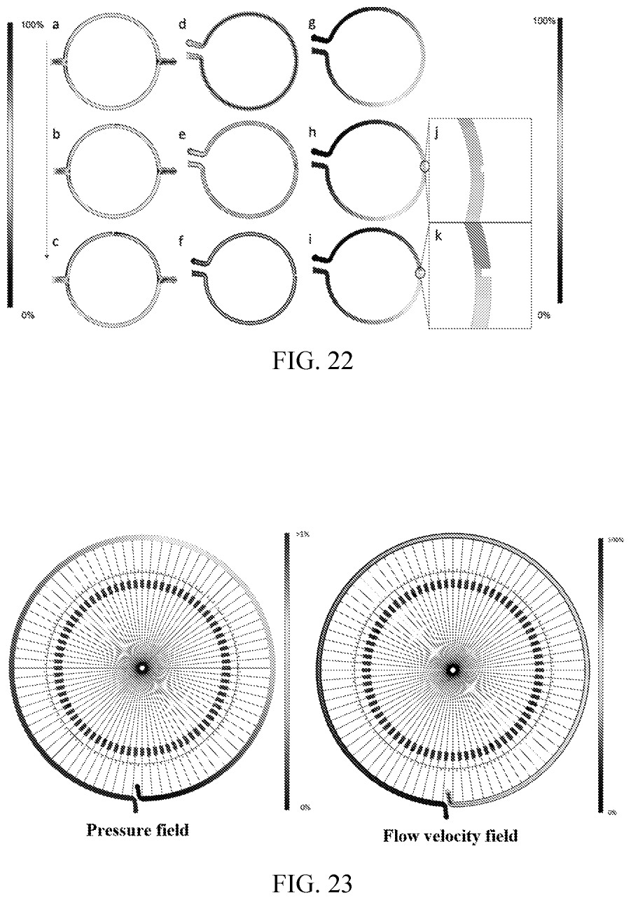

FIG. 21 is fluid simulation data of the integrated chip containing 16 drop-maker units as shown in FIG. 7,
wherein A is a three-dimensional pipeline schematic diagram of the chip, B is a partial enlarged view of a drop-maker unit, C is a simplified diagram of channel resistance, D is a hydraulic distribution thermodynamic diagram in the chip structure in Example 10, E and F are partial enlarged thermodynamic diagrams of the corresponding positions in Diagram D respectively, and G is a flow velocity distribution thermodynamic diagram in the chip structure in Example 10, H is a hydraulic pressure distribution thermodynamic diagram in the chip structure according to Comparative Example 3, I and J are partial enlarged thermodynamic diagrams of the corresponding positions in Diagram H, and K is a flow velocity distribution thermodynamic diagram in the chip structure involved in Comparative Example 3, L is a quantitative diagram of the hydraulic pressure distribution in Diagrams D and H, and M is a quantitative diagram of the flow velocity distribution in Diagrams G and K;

FIG. 22 is fluid simulation data of different washing channel configurations;
where a-c are flow velocity distribution thermodynamic diagrams of the washing channel structure according to Comparative Example 4 under different clogging conditions, d-f are flow velocity distribution thermodynamic diagrams of the washing channel structure under different clogging conditions according to Example 11, g-i are hydraulic pressure distribution thermodynamic diagrams of the washing channel structure under different clogging conditions according to Example 11, and j and k are the local enlarged thermodynamic diagrams of clogging portions of h and i, respectively; and

FIG. 23 is fluid simulation data of the of pressure field and flow field of the integrated chip containing 80 drop-maker units as shown in FIG. 7.

### DETAILED DESCRIPTION OF THE EMBODIMENTS

The microfluidic chip disclosed in the embodiments of the present invention can prepare cell-carried microgel particles of various hydrogel materials in a continuous and stable manner, taking the preparation of hydrogel-based polymers as an example, by combining a parallel and centrosymmetric integration method, and can directly complete washing, demulsification and direct separation of products in the chip. Specific implementations of the present investment will be further described in detail below in conjunction with the accompanying drawings and particular embodiments.

The present invention discloses an integrated microfluidic chip, where at least two drop-maker units are arranged on a substrate, and it also includes a plurality of liquid phase input modules and a washing output module, the liquid phase input modules can be classified as disperse phase distribution unit and continuous phase distribution unit according to the types of liquid phases conveyed inside the liquid phase input modules, the classification of the liquid phase input modules is only associated with the types of the liquid phases inside the liquid phase input modules, but is irrelevant to the relative position in the chip. Therefore, the liquid phase input position in a channel in actual production can be changed randomly, so as to realize the objective of changing the droplet production method according to actual needs and improving the flexibility of chip use. A plurality of distribution modules must include a continuous phase distribution module and one or more dispersed phase distribution modules, and the relative position of each dispersed phase distribution module is also not fixed; the liquid phase input modules are provided with their respective sample injection ports, the washing output module is provided with a washing phase input channel and a product output channel at the same time, and each drop-maker unit is connected to all the liquid phase input modules and the washing channels; there are at least two liquid phase input modules, and at least one substrate containing the liquid phase input module and the corresponding conveying pipelines can be additionally added when there are special preparation needs for microgels; and the continuous phase, the dispersed phase and the washing phase are injected by one or some modes of an injection pump, a peristaltic pump, a pneumatic pump and a hydraulic pump.

According to FIG. 1 or 2, after a liquid phase is pumped into a liquid phase input port 1 (A-1) in the substrate A, the liquid phase is controlled by a resistance control unit 2 (A-2) in a liquid phase input module, after the liquid phase is output from an output port 3 (A-3) of the liquid phase input module, the liquid phase runs through the substrate A and is injected into an input port 4 (B-4) of each drop-maker unit on the substrate B, and then is injected into an emulsification channel 6 (B-6) of the drop-maker unit; similarly, the liquid phase injected into a liquid phase input port 1 (C-1) of other substrate (for example: substrate C) is injected into each drop-maker unit on the substrate B through the resistance control unit 2 and output ports 3 (C-2, C-3) at an equal flow velocity; where the liquid phase in the hydrogel prepolymer phase containing cells or other carrying phases can always maintain a relatively high flow velocity under the control of the distribution resistance structure, thereby ensuring that the carrying phases can stably flow in the hydrogel prepolymer phase without clogging.

In an emulsification channel 6 (B-6) of the drop-maker unit, incompatible liquid phases are mutually fused and sheared at a constant flow rate after passing through a pipeline intersection, so that the emulsification of droplets with stable particle size distribution is realized, and then crosslinking solidification of microgels is induced through oil phase or exogenous crosslinking stimulation, so as to realize the embedding of cells or other carrying phases.

At the downstream of the drop-maker unit, microparticles enter a local resistance control unit 8 (B-8) after running through a standard channel of a certain distance, namely an output channel 7 (B-7), taking an enlarged cavity as an example, due to the size of the enlarged cavity is different from that of a common channel to some extent, microgels then expand to be spherical, and are further solidified and shaped, and continue to migrate in the enlarged cavity; meanwhile, the flow rate of the liquid phase remains at a relatively high level due to the size limitation, so that the microgels can be prevented from being clogged in the channel, so as to keep the flow channel clear.

A washing channel 9 (B-9) is annularly arranged in a unidirectional way, and enlarged cavities at the downstream of the drop-maker units are equidistantly arranged on the inner ring of the washing channel. The washing phase is directly pumped in from a washing phase input port 10 (B-10) and is contacted with the two-phase emulsion discharged from the enlarged cavities in the washing channel, and demulsification and hydrogel separation are realized through the characteristics of a washing agent or a surfactant. Meanwhile, the flow of the multiphase formed by the washing phase and the discharged emulsion still keeps the flow rate in the washing channel, so that the flow rate can still ensure the clear in the entire washing tank even if the size of the washing channel is far greater than that of the standard channel and the enlarged cavity. Further, due to the washing channel has a relatively large size, its internal flow resistance is much smaller than that of the standard channel, therefore, its impact on the resistance downstream of each of the different drop-maker units can be ignored, and finally the two-phase liquid phase containing the product is collected by a product output channel 11 (B-11).

Substrates A, B, C of the chip may be made of one or a mixture of more of glass, silicon, metal and polymer, where the polymer may be one or more of polydimethylsiloxane (PDMS), polymethyl methacrylate (PMMA), engineering plastics (PC), cyclic olefin copolymer (COC), and polyethylene terephthalate (PET), and the substrates are encapsulated by one or more of thermal pressing, adhesive bonding, laser welding, ultrasonic welding, bolt butting, anodic bonding, and plasma bonding.

Taking the preparation of single-component hydrogel particles with only two layers of A and B as an example, the application method of the integrated microfluidic chip is as follows:
1) Liquid phases are pumped into a chip through all sample injection ports, where the hydrogel prepolymer is introduced into the layer A, the continuous oil phase is introduced into the layer B, the washing phase is introduced into the washing channel, and the product output channel is connected to a receiving container;
2) After all liquid phases are stably pumped, the two phases A and B run through a liquid phase input module and then are injected into the drop-maker units, so that the aqueous phase is sheared by the oil phase in the cross-shaped emulsification channel of the drop-maker unit, the emulsification is thus realized, and meanwhile, the cross-linking agent in the oil phase induces the cross-linking of the hydrogel;
3) After leaving the emulsification channel, droplets pass through the output channel, enter the enlarged cavities and are completely stretched into spherical shapes, and are subjected to final crosslinking reaction to realize shaping;
4) The droplets enter the washing channel from the enlarged cavities, are subjected to spontaneous or induced demulsification after contacting with a washing phase in the washing channel, and enter the washing phase to realize elution of hydrogel; and
5) The washing solution containing the microgels and part of incompletely eluted emulsion directly leave the chip through the outlet, and the final washing process is then completed in the pipeline.

It can be seen therefrom that the integrated microfluidic chip disclosed in the present invention features simple supporting equipment and strong structural adjustability, and can adapt to the preparation of different types of hydrogel; the fluid dynamics of liquid phase is used to keep the channel clear and the droplets formation; the washing phase is introduced to keep the washing channel clear and demulsify the water-oil emulsion; an annular integration mode is adopted to realize consistent pipeline resistances among the channels; and a parallel integration mode is adopted to realize the collection of hydrogel under the condition of minimal impact on the drop-maker units. The present invention integrates a large number of drop-maker units into one chip, so that the production time of the microgels is greatly shortened and the production flow is simplified under the condition of keeping the particle size distribution of the microgels, thereby providing an efficient platform for the production of cell-carried microgels or other microgels with carrying phases.

The following embodiments are only used to further illustrate the present invention in detail, and are not intended to limit the present invention in any way.

### Example 1 Preparation of Microgels Carrying MSC Cells with a Chip of Multilayer Structure Integrating 80 Drop-Maker Units

Cell culture: taking the culture of mouse mesenchymal stem cell (MSC) as an example, the proliferation medium is composed of α-minimum Eagle's medium (α-MEM), 10% fetal bovine serum (FBS, Gibco), and the culture conditions are 37° C., 95% relative humidity and 5% CO₂. The cell culture medium was changed after every two days. Before being used, cells were washed with phosphate buffered saline (PBS), placed in trypsin/EDTA solution for 5 minutes, and suspended in the culture medium for standby.

The chip shown in FIG. 7 was used to prepare microgels. An alginic acid prepolymer of sodium alginate with a final concentration of 1%, calcium ethylenediaminetetraacetic acid (Ca-EDTA) with a final ion concentration of 50 mM and MSC cell concentration of 10⁶/ml was configured with an α-MEM medium, which was taken as an aqueous phase and connected to the sample injection port on the substrate A and pumped thereinto at a flow rate of 10 ml/h, and finally entered the drop-maker unit on the substrate B after passing through the resistance control unit; a solution of acetic acid with a final concentration of 2‰ and 5% of perfluorooctyl alcohol was configured with a fluorocarbon oil (HFE7100), which was taken as an oil phase and connected to the sample injection port on the substrate B and pumped thereinto at a flow rate of 80 ml/h, and entered the drop-maker unit on the substrate B after passing through the resistance control unit; and a 4-hydroxyethyl piperazine ethanesulfonic acid (HEPES) solution with a final concentration of 5 mM configured with an α-MEM medium was taken as a washing phase and connected to a washing phase inlet port on the substrate B and pumped thereinto at a flow rate of 120 ml/h, so that entered the washing channel. Local adjustment was made to make all channels stably generating droplets, and the droplet production status in the chip is shown in FIG. 9(a-i); a mixed liquid of the product output channels of the substrate B was collected, after standing for stratification, microgels were distributed at a bottom layer of the upper aqueous phase, the product was then obtained by separating the aqueous phase, and the phase separation state is shown in FIG. 10. The cell-free product fluorescence image is shown in FIG. 11, with an average particle size being 108.11 μm and a difference in particle size distribution being 3.6%.

The cytotoxicity of the block copolymer surfactant system and the metastable emulsion preparation system was investigated by using the Live/Dead fluorescence staining (LIVE/DEAD assay). 2 mM calcein (a green fluorescent dye used to mark living cells) and 4 mM propidium iodide (a red fluorescent dye used to mark dead cells) were added into a microgel suspension and incubated for 20 minutes, the results were then observed by using a confocal laser scanning microscope, and the results are shown in FIG. 12. The cell survival rate is 95.36%, indicating that the method features extremely high biocompatibility.

### Comparative Example 1 Preparation of Microgels Carrying 3T3 Cells in a Single Drop-Maker Unit

The chip structure shown in FIG. 13 was used to prepare microgels. Sodium alginate and Ca-EDTA were dissolved in deionized water to prepare an alginic acid prepolymer solution with sodium alginate content of 1 w/v %, calcium ion with a final concentration of 50 mM and MSC cell concentration of 10⁶/ml as an aqueous phase, and was input from a first input channel at a flow rate of 0.1 ml/h. A solution of acetic acid with a final concentration of 1‰ configured with an HFE7100 and 5% of perfluorooctyl alcohol were taken as an oil phase and input from a second input channel at a flow rate of 1 ml/h. An HEPES solution with a final concentration of 5 mM configured with an α-MEM medium was taken as a washing phase and input from a third input channel at a flow rate of 1 ml/h. Adjustment was made to make all channels stably generating droplets, a mixed liquid of the product output channels was collected, after standing for stratification, microgels were distributed at a bottom layer of the upper aqueous phase, and the product was then obtained by separating the aqueous phase. The cell survival rate of the product was 97.55%, and the cell culture and fluorescence detection methods were the same as those in Example 1. The production throughput of cell-carried microgels is two orders of magnitude smaller than that of Example 1, indicating a high production throughput of the method in the present invention.

### Comparative Example 2 Preparation of Microgels with a Chip of Multilayer Structure Integrating 16 Drop-Maker Unit and Having a Washing Channel with a Symmetrical Structure

The chip having a washing channel with a symmetrical structure as shown in FIG. 3 was used to prepare microgels. Sodium alginate and Ca-EDTA were dissolved in deionized water to prepare an alginic acid prepolymer solution with sodium alginate content of 1 w/v % and calcium ion with a final concentration of 50 mM as an aqueous phase, and was input from a first input channel at a flow rate of 1.6 ml/h. A solution of acetic acid with a final concentration of 1‰ configured with an HFE7100 and 5% of perfluorooctyl alcohol were taken as an oil phase and input from a second input channel at a flow rate of 16 ml/h. An HEPES solution with a final concentration of 5 mM configured with ultrapure water was taken as a washing phase and input from a third input channel at a flow rate of 16 ml/h. Adjustment was made to make all channels stably generating droplets, and microgels production was continuously performed. The liquid phase flow state at the end of the washing channel is shown in FIG. 14(A). After the production lasted for 15 minutes, the liquid phase flow state at the end of the washing channel is shown in FIG. 14(B), it can be seen that the washing channel at one side has been completed clogged due to local accumulation of microgels in the channel. The liquid phase flow states at the end of the washing channel at the beginning of the production, as well as 30 minutes later, of Example 1 are shown in FIGS. 14(C) and (D), indicating that a stable flow can be maintained for a long time, thus proving that the washing channel structure is suitable for the production of microgels under the structural system.

### Example 2 Preparation of Microgels Having Core-Shell Structure and Carrying Nanoparticles with a Chip of Multilayer Structure Integrating 16 Drop-Maker Unit

The chip shown in FIG. 2 was used to prepare microgels. An alginic acid prepolymer solution of sodium alginate with a final concentration of 1%, 0.1% of fluorescence-modified nanoparticles and calcium ethylenediaminetetraacetic acid (Ca-EDTA) with a final ion concentration of 50 mM was configured with ultrapure water, which was taken as a shell phase and connected to the sample injection port on the substrate A and pumped thereinto at a flow rate of 1.6 ml/h; pure water was taken as a core phase and connected to the sample injection port at the middle of the substrate B through a horizontal input channel and was pumped thereinto at a flow rate of 1.6 ml/h, so as to enter the sample injection port at the middle of the substrate B; 5% of perfluorooctyl alcohol solution configured with an HFE7100 was taken as an oil phase and connected to the sample injection port on the substrate C and pumped thereinto at a flow rate of 16 ml/h; acetic acid with a final concentration of 2‰ configured with an HFE7100 was taken as a cross-linked initiation phase and connected to the washing phase input channel on the substrate B and pumped thereinto at a flow rate of 32 ml/h. Local adjustment was made to make all channels stably generating droplets, and the droplet production status in the chip is shown in FIG. 15; the liquid phase flow states at other positions are shown in FIGS. 9(a), (b), and (c). A mixed liquid of the product output channels of the substrate B was collected, after standing for stratification, microgels were distributed at a bottom layer of the upper aqueous phase, the product was then obtained by separating the aqueous phase, and the phase separation state is shown in FIG. 16, it can be seen that the alginate hydrogel shell has fluorescence, indicating that the method of the present invention may continuously and stable prepare the microgel particles with core-shell structure in a high-throughput manner.

### Example 3 Preparation of Janus Microgels and Carrying Different Cells with a Chip of Multilayer Structure Integrating 16 Drop-Maker Units

The chip shown in FIG. 2 was used to prepare microgels, where the drop-maker unit was the structure shown in FIG. 5(B). An alginic acid prepolymer solution of sodium alginate with a final concentration of 1%, calcium ethylenediaminetetraacetic acid (Ca-EDTA) with a final ion concentration of 50 mM and NIH3T3 cells (mouse embryonic fibroblast cell line) with a concentration of 10⁶/ml was configured with Dulbecco's modified eagle medium (DMEM), which was taken as an aqueous phase No. 1 and connected to the sample injection port on the substrate A and pumped thereinto at a flow rate of 1.6 ml/h; an alginic acid prepolymer solution of sodium alginate with a final concentration of 1%, calcium ethylenediaminetetraacetic acid (Ca-EDTA) with a final ion concentration of 50 mM and Hela cells with a concentration of 10⁶/ml was configured with DMEM, which was taken as an aqueous phase No. 2 and connected to the sample injection port at the middle of the substrate B and pumped thereinto at a flow rate of 1.6 ml/h; a solution of acetic acid with a final concentration of 1‰ and 5% of perfluorooctyl alcohol solution was configured with an HFE7100, which was taken as an oil phase and connected to the sample injection port on the substrate C and pumped thereinto at a flow rate of 16 ml/h; an HEPES solution with a final concentration of 5 mM configured with an HFE7100 was taken as a washing phase and connected to the washing phase input channel on the substrate B and pumped thereinto at a flow rate of 24 ml/h. Local adjustment was made to make all channels stably generating droplets, and a mixed liquid of the product output channels of the substrate B was collected, after standing for stratification, microgels were distributed at a bottom layer of the upper aqueous phase, the product was then obtained by separating the aqueous phase. The cell-free product fluorescence image is shown in FIG. 17, it can be seen that the volume ratio of red to green hemispheres is 1:1, indicating that the method of the present invention may continuously and stable prepare the Janus microgel in a high-throughput manner.

### Example 4 Preparation of Microgels Carrying Rat Mesenchymal Stem Cell (MSC) with a Chip of Multilayer Structure Integrating 16 Drop-Maker Units

The chip shown in FIG. 2 was used to prepare microgels. An aqueous solution of PEGDA with a final concentration of 1% and 1% of photoinitiator 2959 was configured with an α-MEM medium, which was taken as an aqueous phase No. 1 and connected to the sample injection port on the substrate A and pumped thereinto at a flow rate of 1.6 ml/h; a prepolymer solution of PEGDA with a final concentration of 1% and MSC cell concentration of 10⁶/ml was configured with an α-MEM medium, which was taken as an aqueous phase No. 2 and connected to a sample injection port on the substrate B and pumped thereinto at a flow rate of 1.6 ml/h; a solution of PFPE-PEG-PFPE with a final concentration of 1% configured with HFE7100 was taken as an oil phase and connected to a sample injection port on the substrate C and pumped thereinto at a flow rate of 16 ml/h; the washing phase input channel was blocked, the product output channel was connected by a PE pipe and directly irradiated with 356 nm ultraviolet light, the resulting product was added with an HFE7100 solution containing 20% of perfluorooctyl alcohol, and a pure culture medium was added in the upper layer thereof for washing. After standing for stratification, cell-carried microgels were distributed at a bottom layer of the upper aqueous phase, and the product was then obtained by separating the aqueous phase. The cell-free product image is shown in FIG. 18, with an average particle size being 56.36 μm and a difference in particle size distribution being 2.3%, indicating that the method of the present invention may adapt to the high-throughput continuous and stable production of photo-initiating hydrogel particles.

### Example 5 Preparation of Smaller/Larger Size Microgels with a Chip of Multilayer Structure Integrating 16 Drop-Maker Units

As shown in FIG. 7, a square chip provided with a production channel unit with a cross section side length of 10 μm and 500 μm respectively was used to prepare microgels. An alginic acid prepolymer of sodium alginate with a final concentration of 1% and calcium ethylenediaminetetraacetic acid (Ca-EDTA) with a final ion concentration of 50 mM was configured with DMEM, which was taken as an aqueous phase and connected to the sample injection port on the substrate A and pumped thereinto at a flow rate of 1.6 ml/h, and finally entered the drop-maker unit on the substrate B after passing through the resistance control unit; a solution of acetic acid with a final concentration of 2‰ and 5% of perfluorooctyl alcohol was configured with an HFE7100, which was taken as an oil phase and connected to the sample injection port on the substrate B and pumped thereinto at a flow rate of 16 ml/h, and entered the drop-maker unit after passing through the resistance control unit; an HEPES solution with a final concentration of 5 mM configured with an DMEM medium was taken as a washing phase and connected to the washing phase input channel on the substrate B and pumped thereinto at a flow rate of 16 ml/h, and entered the washing channel. Local adjustment was made to make all channels stably generating droplets, and the droplet production status in the chip is shown in FIG. 9(a-ii); a mixed liquid of the product output channels of the substrate B was collected, after standing for stratification, microgels were distributed at a bottom layer of the upper aqueous phase, the product was then obtained by separating the aqueous phase. The obtained product has an average particle size being 18.11 μm and 805.65 μm, respectively, and a difference in particle size distribution being 5.3% and 4.4%, respectively, indicating that the method of the present invention may adapt to produce microgel particles of different sizes.

### Example 6 Preparation of Microgels at Different Flow Rates with a Chip of Multilayer Structure Integrating 16 Drop-Maker Units

As shown in FIG. 7, a square chip provided with a production channel unit with a cross section side length of 50 μm was used to prepare microgels. An alginic acid prepolymer of sodium alginate with a final concentration of 1% and calcium ethylenediaminetetraacetic acid (Ca-EDTA) with a final ion concentration of 50 mM was configured with ultrapure water, which was taken as an aqueous phase and connected to the sample injection port on the substrate A and pumped thereinto at a flow rate of 1.6 ml/h, 2.4 ml/h, 3.2 ml/h, 4 ml/h, 4.8 ml/h, 6 ml/h and 8 ml/h, respectively, and finally entered the drop-maker unit on the substrate B after passing through the resistance control unit; a solution of acetic acid with a final concentration of 2‰ and 5% of perfluorooctyl alcohol was configured with an HFE7100, which was taken as an oil phase and connected to the sample injection port on the substrate B and pumped thereinto at a flow rate of 16 ml/h, and entered the drop-maker unit after passing through the resistance control unit; an HEPES solution with a final concentration of 5 mM configured with an DMEM medium was taken as a washing phase and connected to the washing phase input channel on the substrate B and pumped thereinto at a flow rate of 16 ml/h, and entered the washing channel. Local adjustment was made to make all channels stably generating droplets, and a mixed liquid of the product output channels of the substrate B was collected, after standing for stratification, microgels were distributed at a bottom layer of the upper aqueous phase, the product was then obtained by separating the aqueous phase. The particle size distribution of the obtained product is shown in FIG. 19, indicating that the method of the present invention may produce microgel particles of different sizes under conditions of different flow rates.

### Example 7 Preparation of Microdroplets with a Chip of Multilayer Structure Integrating 16 Drop-Maker Units

he chip shown in FIG. 7 was used to prepare droplets. Ultrapure water was taken as an aqueous phase and connected to the sample injection port on the substrate A and pumped thereinto at a flow rate of 1.6 ml/h, and finally entered the drop-maker unit on the substrate B after passing through the resistance control unit; a solution of PFPE-PEG-PFPE with a final concentration of 1% configured with HFE7100 was taken as an oil phase and connected to the sample injection port on the substrate B and pumped thereinto at a flow rate of 16 ml/h, and entered the drop-maker unit after passing through the resistance control unit; and the washing phase inlet on the substrate B was blocked. Local adjustment was made to make all channels stably generating droplets, a mixed liquid of the product output channels on the substrate B was collected, and after standing for stratification, the upper layer was separate to obtain the product. The particle size distribution of the product after adjusting the flow ratio is shown in FIG. 20, when the flow ratio of the aqueous phase to the oil phase is less than 2:5 for droplet production, droplets with a particle size distribution of less than 3% may be obtained; and when the flow ratio of the same is greater than 3:5, the size distribution range of droplets is obviously wider, indicating that stable droplets are unable to be formed.

### Example 8 Preparation of Gelatin Particles with a Chip of Multilayer Structure Integrating 16 Drop-Maker Units

The chip shown in FIG. 7 was used to prepare gelatin particles. At 40° C., a gelatin solution with a final concentration of 10% configured with ultrapure water was taken as an aqueous phase and connected to the sample injection port on the substrate A and pumped thereinto at a flow rate of 1.6 ml/h, and finally entered the drop-maker unit on the substrate B after passing through the resistance control unit; a solution of PFPE-PEG-PFPE with a final concentration of 1% configured with HFE7100 was taken as an oil phase and connected to the sample injection port on the substrate B and pumped thereinto at a flow rate of 16 ml/h, and entered the drop-maker unit after passing through the resistance control unit; the chip is overall placed in an environment of 37° C., and the washing phase inlet on the substrate B was blocked. Local adjustment was made to make all channels stably generating droplets, a mixed liquid of the product output channels on the substrate B was collected and stood for stratification in ice-water bath; after the upper layer was separate, an 20% PFO contained HFE7100 solution with an equal volume is added, and equal volume ultrapure water is added, the product may be obtained after oscillation, indicating that the method of the present invention may continuously and stable prepare the temperature-sensitive hydrogel particles.

### Example 9 Preparation of Plastic Particles with a Chip of Multilayer Structure Integrating 80 Drop-Maker Units

Chips shown in FIGS. 1 and 7 were used to prepare polystyrene plastic microparticles. Polystyrene was dissolved in toluene to prepare a toluene solution with a polystyrene mass fraction of 20%, which was taken as the oil phase and input from the first input channel at a flow rate of 20 ml/h. Polyvinyl alcohol was dissolved in water to prepare an aqueous solution with a polyvinyl alcohol mass fraction of 10%, which was taken as an aqueous phase and input from the second input channel at a flow rate of 100 ml/h. The washing phase inlet on the substrate B was blocked. Adjustment was made to make all channels stably generating droplets, and a mixed liquid of the product output channels was collected and placed in a constant temperature drying oven after standing for stratification. After the toluene volatilized, plastic particles were distributed on the surface of the aqueous phase, and the product can be obtained after separation, indicating that the method of the present invention may continuously and stably prepare plastic microparticles in a high throughput way.

### Example 10 Computational Fluid Dynamics Simulation of a Chip with AB Two-Layer Structure Integrated with 16 Drop-Maker Units and Containing a Resistance Control Unit Structure

A two-dimensional structure vector diagram of the chip channel was drawn by using Auto CAD (Autodesk Inc.), and a channel region was selected and a micro-channel two-dimensional structure model was constructed after importing into COMSOL Multiphysics (COMSOL Co.). A liquid phase material and a liquid phase input port were selected, a flow velocity (equaling to the flow velocity of actual production) was set, the model was gridded, and a steady-state fluid simulation was performed to obtain a flow field and pressure field simulation diagram under the given conditions. The hydraulic field in the channel and its quantification (FIGS. 21(H), (I), (J), (L)) show that the overall resistance of the washing channel is less than 1% of the fluidic resistance in each drop-maker unit, meeting the integration criteria, therefore, the difference in flow velocity among channels (FIGS. 21(K), (M)) is small. Combining with the results of Example 9, it indicates that the method of the present invention can produce microdroplets with uniform particle size distribution.

### Comparative Example 3 Computational Fluid Dynamics Simulation of a Chip with AB Two-Layer Structure Integrated with 16 Drop-Maker Units and Containing No Resistance Control Unit Structure

A two-dimensional structure vector diagram of a channel without a chip resistance control unit structure was drawn by using Auto CAD (Autodesk Inc.), and a channel region was selected and a micro-channel two-dimensional structure model was constructed after importing into COMSOL Multiphysics (COMSOL Co.). A liquid phase material and a liquid phase input port were selected, a flow velocity (equaling to the flow velocity of actual production) was set, the model was gridded, and a steady-state fluid simulation was performed to obtain a flow field and pressure field simulation diagram under the given conditions. The hydraulic field in the channel and its quantification (FIGS. 21(D), (E), (F), (L)) show that the overall resistance of the washing channel is greater than 3% of the fluidic resistance in each drop-maker unit, failing to meet the integration criteria, therefore, the difference in flow velocity among channels (FIGS. 21(G), (M)) is larger, and microdroplets with uniform particle size distribution cannot be produced.

### Example 11 Computational Fluid Dynamics Simulation of a Annular Washing Channel

A two-dimensional structure vector diagram of the chip channel (as shown in FIG. 22(d)) was drawn by using Auto CAD (Autodesk Inc.), and a channel region was selected and a micro-channel two-dimensional structure model was constructed after COMSOL importing into Multiphysics (COMSOL Co.). A liquid phase material and a liquid phase input port were selected, a flow velocity (equaling to the flow velocity of actual production) was set, the model was gridded, and a steady-state fluid simulation was performed to obtain a flow field and pressure field simulation diagram under the given conditions. Results of the flow field in the channel (FIGS. 22(d)-(k)) indicate that local pressure (FIGS. 22(j) and (k)) and local flow velocity (FIG. 22(f)) at the clogged portion increase sharply when fine microgels deposit in the channel, while the clogging caused by deposit of microgels does not have high structural strength, it is thus easily dispersed by a mixed liquid phase under the conditions of high flow velocity and high pressure, thereby solving the problem of local clogging. The flow state of the liquid phase in the actual production is stated in Comparative Example 2, local clogging in the channel can be directly broken down by the washing phase with high flow rate velocity and high hydraulic pressure, so that stable operation of the liquid phase in the washing channel can be maintained.

### Comparative Example 4 Computational Fluid Dynamics Simulation of a Parallel and Symmetrical Washing Channel

A two-dimensional structure vector diagram of the chip channel (as shown in FIG. 22(a)) was drawn by using Auto CAD (Autodesk Inc.), and a channel region was selected and a micro-channel two-dimensional structure model was constructed after importing into COMSOL Multiphysics (COMSOL Co.). A liquid phase material and a liquid phase input port were selected, a flow velocity (equaling to the flow velocity of actual production) was set, the model was gridded, and a steady-state fluid simulation was performed to obtain a flow field and pressure field simulation diagram under the given conditions. Results of the flow field in the channel (FIGS. 22(a)-(c)) indicate that the fluid resistance of one side increases significantly when such side suffers a small amount of uncontrollable accumulation, which results in a decrease in the flow velocity distributed on the partially clogged side, and the decreased flow rate and flow velocity further increase the probability of clogging of microgels in the channel on the side, after such a repeated vicious cycle, the channel on the side will be inevitably clogged completely, which in turn affects the flow distribution of the liquid phase input on the other side and seriously affects the overall quality of the microgel product. The flow state of the liquid phase in the actual production is stated in Comparative Example 2, and local clogging in the channel can cause complete clogging of the whole channel in a short time, therefore, the symmetrical parallel washing structure is unsuitable for eluting and collecting the solidified microgels in the channel.

### Example 12 Computational Fluid Dynamics Simulation of a Chip with AB Two-Layer Structure Integrated with 80 Drop-Maker Units and Containing a Resistance Control Unit Structure

A two-dimensional structure vector diagram was drawn by using Auto CAD (Autodesk Inc.), and a channel region was selected and a micro-channel two-dimensional structure model was constructed after importing into COMSOL Multiphysics (COMSOL Co.). A liquid phase material and a liquid phase input port were selected, a flow velocity (equaling to the flow velocity of actual production) was set, the model was gridded, and a steady-state fluid simulation was performed to obtain a flow field and pressure field simulation diagram under the given conditions, as shown in FIG. 23. The hydraulic field in the channel and its quantification show that the overall resistance of the washing channel is less than 1% of the fluidic resistance in each drop-maker unit, meeting the integration criteria. Combining with the results of Example 1, it indicates that the difference in flow velocity among channels is small, and microdroplets with inform particle size distribution can be produced.

For any skilled in the art, without departing from the scope of the technical solution of the present invention, many possible changes and modifications can be made to the technical solution of the present invention by using the technical contents disclosed above, or modified into equivalent embodiments with equivalent changes. Therefore, any simple modification, equivalent change and modification made to the above embodiments according to the technical essence of the present invention without departing from the contents of the technical solution of the present invention shall still belong to the protection scope of the technical solution of the present invention.

1. A multi-channel integrated microfluidic chip, comprising at least two layers of channel structure, at least two liquid phase input channels, at least two drop-maker units and a collection channel;
each layer of channel structure provided with a liquid phase input channel, wherein one of the layers of channel structure is provided with drop-maker units, and the collection channel is contained in one of the layers of channel structure or cross through the multiple layers of channel structure;
each liquid phase input channel comprising at least one liquid phase input port (1), wherein the liquid phase input port (1) is connected to at least one resistance control unit (2), and each resistance control unit corresponding to one output port (3);
the drop-maker unit comprising an input port (4), a liquid phase input channel (5), an emulsification channel (6), an output channel (7) and a local resistance control unit (8), wherein the output ports (3) on the different layers of channel structure correspond to the input ports (4) on the same and are communicated with each other through a microfluidic channel, and the resistance control units (2) on the same layer of channel structure are directly connected to the emulsification channel; and
the collection channel comprising a washing channel (9), a washing phase input port (10) and a product output port (11).

2. The multi-channel integrated microfluidic chip according to claim 1, wherein:
when the number of input liquid phases is 2, the two liquid phases are input through the liquid phase input ports (1) of the outermost layer channel structure of the multi-channel integrated microfluidic chip, respectively; when the number of the input liquid phases is greater than or equals to 3, the liquid input ports of the layers other than the outermost layer are respectively connected to the side surface of the chip through the horizontal input channels (12) to input liquid phases.

3. The multi-channel integrated microfluidic chip according to claim 1, wherein the liquid phase input channels and the drop-maker units in the chip are arranged in a centrosymmetric manner with the liquid phase input port as a center, and liquid input ports (1) of all liquid phase input channels are located on the same longitudinal axis.

4. The multi-channel integrated microfluidic chip according to claim 1, wherein a structure of the resistance control unit (2) is selected from one or a combination of some of a mesh groove, an annular groove and an S-shaped channel structure, and a structure of the local resistance control unit (8) is selected from one or some of a local bayonet structure, an S-shaped channel structure or an enlarged cavity structure.

5. The multi-channel integrated microfluidic chip according to claim 1, wherein a structure of the emulsification channel (6) in the drop-maker unit is selected from one or some of a flow-focusing structure, a T-junction structure and a co-flow structure.

6. The multi-channel integrated microfluidic chip according to claim 1, wherein a channel in the drop-maker unit of the chip has a width ranging from 5 μm to 500 μm and a cross-sectional area of 25 μm² to 10⁶ μm².

7. The multi-channel integrated microfluidic chip according to claim 1, wherein the washing channel is annularly arranged in a unidirectional way, output channels (7) of all the drop-maker units are equidistantly arranged on the inner circumference of the washing channel, the beginning and the end of the washing channel are a washing phase input port (10) and a product output port (11), respectively, and a channel cross-sectional area of the washing channel is more than 10 times of that of the drop-maker unit.

8. A method for preparing monodisperse gel microspheres, wherein the method uses the microfluidic chip according to claim 1 a single or multiple dispersion phases are used as a first fluid, a continuous phase is used as a second fluid, and a washing phase is used as a third fluid; the first fluid and the second fluid enter the emulsification channel in the drop-maker unit through the liquid phase input channel, the first fluid is sheared by the second fluid in the emulsification channel to form droplets and then form microgels to enter a washing output module; when the number of the liquid phase of the first fluid is greater than or equal to 2, all the liquid phases are combined into one phase in the channel and then enter the emulsification channel; the third fluid cleans the two-phase emulsion in a washing module, the flow velocity in the washing module is maintained to prevent micro gel particles from aggregating and clogging, and droplets of the first fluid form the monodisperse gel microspheres through an internal crosslinking of macromolecules.

9. The method for preparing monodisperse gel microspheres according to claim 8,
wherein the first fluid is a bioactive substance suspended in the dispersed phase; when multiple carrying is performed, the carrying method of different substances is selected from one of suspended in the same dispersed phase, suspended in a plurality of groups of pre-differentiated dispersed phases, suspended in a plurality of groups of dispersed phases difficult to be mutually soluble in a same solvent, and suspended in a mutually soluble multi-dispersed phases, wherein the bioactive substances are selected from one or more of living cells, drugs, nucleic acids, proteins, flavors, nanoparticles and quantum dots;
a carrier macromolecule in the first fluid comprises one or more of a hydrogel prepolymer and a crosslinkable macromolecule prepolymer; a curing manner of the prepolymer in the first fluid comprises one or more of chemical crosslinking, photo-crosslinking, temperature-sensitive curing and phase separation;
the second fluid comprises at least one surfactant;
at least one phase of the first fluid, the second fluid and the third fluid contain at least one prepolymer crosslinking initiator; a crosslinking initiator is not needed when the temperature-sensitive curing is adopted;
when the preparation of the cell-carried microgels is performed, the third fluid is an aqueous phase, the main body thereof is a cell-compatible solvent, and also comprises a pH buffering agent; and
the monodisperse gel microspheres comprise microgel particles, microcapsules/micro-vesicles and multi-cavity microcapsules, with an average particle size being greater than or equal to 5 μm.
